(19) United States
(12) Patent Application Publication
(10) Pub. No.: US 2025/0197075 A1
(43) Pub. Date: Jun. 19, 2025

(54) Dispenser device with Anti-Leak and Anti-Humidity Features

(71) Applicant: Javier Julia, Madrid (ES)

(72) Inventor: Javier Julia, Madrid (ES)

(21) Appl. No.: 18/542,634

(22) Filed: Dec. 16, 2023

(51) Int. Cl. B65D 51/30; A61M 15/08; B65D 41/04; B65D 53/02; B65D 83/06

(52) U.S. Cl. CPC B65D 51/30; A61M 15/08; B65D 41/0407; B65D 53/02; B65D 83/06; A61M 2202/064; B65D 2251/0015; B65D 2251/0078; B65D 2251/205

(57) ABSTRACT

The present invention relates to a novel dispenser device designed for the efficient and controlled delivery of powdered substances. It comprises an aluminium housing that is both lightweight and durable, ensuring ease of handling and robust protection for the internal components. An upper cap with an anti-leak design snugly fits onto the housing, effectively preventing substance leakage. The inhaler features a mechanically operated side dispenser knob, which activates an internal mechanism to dispense precise doses of substance. Additionally, a unique bottom cap contains a refillable compartment with silica particles, providing effective moisture control to preserve the substance's integrity.

### TECHNICAL FIELD

This invention pertains to the field of medical devices, with a specific focus on devices designed for nasal administration of powdered substances. It addresses the need for improved delivery mechanisms that maintain the integrity and efficacy of the substance.

### BACKGROUND

In the realm of medical devices, particularly dispenser devices for powdered substances, several challenges have persisted over the years, impacting the efficacy and user experience of these devices. The primary issues revolve around substance leakage and the effects of humidity on the substance.

Dispenser devices have long been used for administering substance directly to the nasal passages. However, the primary challenge has been ensuring the precise and controlled release of substance. In powdered form, substances are particularly susceptible to external environmental factors like air and moisture. Leakage of substance is a common problem, leading to inconsistent dosing and potential wastage of substance. This not only affects the cost-effectiveness but also impacts the therapeutic effectiveness of the treatment.

Moreover, powdered substances, when exposed to humidity, tend to clump or aggregate. This not only makes it difficult to dispense the substance but also can alter the substance's properties, potentially leading to reduced effectiveness or even adverse reactions in patients. Maintaining the stability and dryness of the substance is thus crucial.

To address these challenges, various designs and mechanisms have been implemented in dispenser devices. Traditional designs often include features like tight-sealing caps and moisture-resistant materials. However, these solutions have been only partially successful. While caps are designed to prevent substance leakage, they often fail to provide a completely air-tight seal. This is particularly evident in cases where the user may not properly close the cap after each use, leading to air exposure and the risk of substance spoilage. Some inhalers use materials purported to resist moisture. However, these materials often do not address the issue of internal humidity, which can arise from the breath of the user or ambient humidity. Some manufacturers include desiccants in the packaging to absorb moisture. While this can be effective in storage, it does not address the issue during actual use when the substance is most vulnerable to humidity. Furthermore, mechanical dispensing mechanisms aim to provide precise doses of substance. Yet, these systems are often complex, prone to mechanical failure, and do not necessarily prevent the exposure of the substance to air and humidity between uses. Additionally, the effectiveness of these solutions is often user dependent. Improper use or handling of the inhaler can negate the benefits of the design features, leading to the aforementioned issues of leakage and humidity exposure.

Given these limitations, there is a clear need for an improved dispenser device design that effectively addresses both substance leakage and humidity control in a user-friendly and reliable manner. The ideal solution would offer a secure sealing mechanism that ensures the substance remains contained and protected from external air exposure. Additionally, it should incorporate a system to control internal humidity, preserving the substance's integrity throughout its shelf life and during use. This solution should be robust yet simple enough for easy use by the patient, minimizing the reliance on user technique and adherence to ensure the efficacy of the substance delivery. In summary, while existing dispenser devices have made strides in addressing the challenges of substance delivery, they fall short in providing a comprehensive solution that effectively tackles both substance leakage and humidity control. The current invention aims to fill this gap, offering a design that overcomes the limitations of existing methods and enhances the overall effectiveness and reliability of dispenser devices for powdered substances.

### SUMMARY

Embodiments of the present disclosure present technological improvements as solutions to one or more of the above-mentioned technical problems recognized by the inventor in conventional solutions.

The present invention discloses a novel dispenser device designed specifically for dispensing powdered substance, addressing key issues prevalent in existing devices which is substance leakage and humidity-induced deterioration. This invention significantly improves upon the current state of dispenser devices by incorporating a meticulously designed aluminium housing, an advanced anti-leak upper cap, a precise and user-friendly side dispenser knob, and a bottom cap equipped with a silica particle compartment for moisture control. The aluminium housing ensures the device is both lightweight and durable, enhancing portability and ease of handling. The upper cap is engineered to fit snugly onto the housing, creating an effective anti-leak seal. This design feature is crucial in preventing the spillage of substance and in protecting it from external environmental factors. The side dispenser knob, operated mechanically, allows for controlled and accurate dispensing of substance, contributing to consistent dosing and efficacy. Most notably, the bottom cap's unique inclusion of a silica particle compartment addresses the issue of humidity, a common problem in the storage and use of powdered substances. These silica particles absorb moisture, thus maintaining the dryness and stability of the substance. This compartment is designed for ease of use, allowing users to replace silica particles as needed.

Additional aspects, advantages, features and objects of the present disclosure would be made apparent from the drawings and the detailed description of the illustrative embodiments construed in conjunction with the complete specification that will follow.

It will be appreciated that features of the present disclosure are susceptible to being combined in various combinations without departing from the scope of the present disclosure.

### OBJECT OF INVENTION

One of the objects of the present invention is to enhance the overall efficiency of delivering powdered substance through the nasal route, ensuring optimal therapeutic effect.

Another object of the present invention is to create a design that is user-friendly and straightforward to encourage regular and correct use by patients, thereby improving treatment compliance.

Yet another object of the present invention is to tackle prevalent challenges in existing dispenser devices, such as substance wastage due to leakage and decreased efficacy due to humidity.

### BRIEF DESCRIPTION OF DRAWINGS

The summary above, as well as the following description of illustrative embodiments are better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those in the art will understand that the drawings are not to scale. Wherever possible, like elements have been indicated by identical numbers.

Embodiments of the present disclosure will now be described, by way of example only, with reference to the following diagrams wherein:

FIG. 1 depicts an exploded view of a dispenser device as per the disclosed invention.an exemplary apparatus as per the disclosed invention.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the nonunderlined number is used to identify a general item at which the arrow is pointing.

### DESCRIPTION OF EMBODIMENTS

The following description illustrates embodiments of the present disclosure and ways in which they can be implemented. Although some modes of carrying out the present disclosure have been disclosed, those skilled in the art would recognize that other embodiments for carrying out or practicing the present disclosure are also possible.

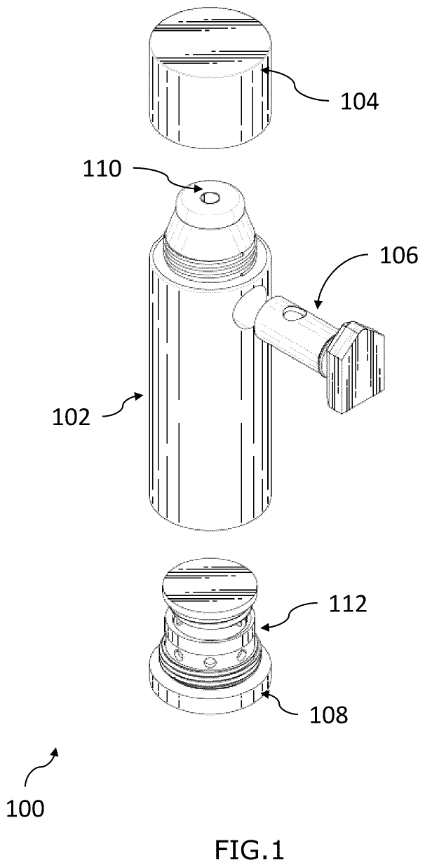

FIG. 1 depicts an exploded view of a dispenser device 100 as per the disclosed invention an exemplary apparatus as per the disclosed invention. The dispenser device 100 comprising a housing body 102, an upper cap 104, a side dispenser knob 106, and a bottom cap 108. In an embodiment, the housing body 102 of the dispenser device 100 is made from aluminium, chosen for its lightweight and durable properties, facilitating ease of handling and portability for users. The upper cap 104 is designed to fit tightly onto the housing body 102, creating an anti-leak seal. This feature is critical in preventing the powdered substance from spilling or being exposed to external elements. The upper cap 104 covers the air intake cavity 110 located at the top of the housing body 102, playing a vital role in the inhaler's anti-leak breathing functionality. Positioned on the side of the housing body 102, the side dispenser knob 106 is integral to the dispenser device's operation. It requires manual twisting to dispense the substance. When twisted, it activates an internal mechanism, presumably a dish or similar component, that accurately meters and releases the prescribed amount of substance for inhalation. Uniquely designed with a compartment 112 for housing silica particles, the bottom cap 108 addresses the issue of humidity. Silica particles are well-known for their moisture-absorbing qualities, ensuring that the powdered substance remains dry and free from clumping. This compartment 112 is designed for easy removal and refilling, allowing users to replace the silica particles as needed.

The core structure of the dispenser device 100 is the housing body 102 made up of aluminium, chosen for its unique combination of lightweight and durability. The housing body 102 stores the powdered substance. Aluminium is an ideal material for medical devices due to its resistance to corrosion and its ability to withstand regular use without significant wear. The housing body's design is ergonomic, allowing for ease of handling and use by patients. Its robust nature ensures that the internal components are well-protected, and the substance contained within is safeguarded from external physical impacts.

The upper cap 104 of the dispenser device 100 is crafted from aluminium, chosen for its lightweight durability and compatibility with the aluminium housing. This choice ensures that the cap is robust enough to withstand regular use, yet not cumbersome for the user to handle. The precision with which the cap fits onto the housing is essential to create an effective seal. This sealing is further enhanced by a specialized mechanism, such as a gasket, made from a flexible and resilient material. This feature is critical in establishing an airtight seal, effectively transforming the cap into a barrier against substance leakage and external contaminants. Functionally, the upper cap's anti-leak feature stands out as its primary role, preventing the escape of powdered substance and ensuring dosage accuracy. The sealing mechanism's ability to create a hermetic environment within the inhaler is essential, not just for preventing wastage, but also for maintaining the purity and efficacy of the substance. Protection against external contamination is equally crucial, as it ensures the substance remains safe and effective for use. Despite its robust sealing capabilities, the upper cap 104 is designed with user accessibility in mind. Features like grooves, ridges, or a textured surface are incorporated to provide a better grip, allowing for easy opening and closing of the upper cap 104, catering especially to users with limited hand strength or dexterity. This ease of use is pivotal in ensuring that patients can access their substance reliably and without struggle. Additionally, by preventing air and moisture ingress, the upper cap 104 aids in preserving the substance's integrity, keeping it in the desired powdered state and protected from environmental factors that could compromise its quality.

The side dispenser knob 106 of the dispenser device 100 ensures precise and consistent substance dispensing. The side dispenser knob 106 serves as the primary interface for the mechanical operation of the dispenser device 100. The knob's surface might be textured or contoured to provide a secure grip, allowing for precise control when being operated. Upon twisting the side dispenser knob 108, the powdered substance is dispensed. In an embodiment, twisting the side dispenser knob 108 triggers the release of powdered substance. The mechanical nature of the side dispenser knob 106 also adds a layer of reliability. It operates independently of external power sources, making the inhaler more dependable and suitable for use in various settings.

The bottom cap 108 of the dispenser device 100, featuring a compartment 112 for silica particles, is designed from materials that complement the rest of the dispenser device 100, ensuring durability and ease of use. The bottom cap 108 must be easy to open yet secure enough to prevent accidental detachment, preserving the integrity of the inhaler's contents. Its design includes a threading mechanism or a snap-fit feature to attach seamlessly to the aluminium housing body 102, providing a sturdy and reliable closure. Within this bottom cap 108 lies a specially designed compartment 112 filled with silica particles. Silica, known for its exceptional hygroscopic properties, plays a vital role in moisture control. By absorbing and retaining moisture, these particles effectively regulate the internal environment of the inhaler, a crucial aspect for preserving powdered substance. This ability to control humidity is essential, as exposure to moisture can lead to substance clumping or degradation, compromising its effectiveness and safety. The design of the silica particle compartment 112 is a model of practicality and user consideration. It is engineered for easy access, allowing users to refill or replace the silica particles as needed. This refillable design not only enhances the inhaler's functionality but also contributes to its sustainability and long-term usability. The compartment 112 must be secure enough to hold the particles in place while allowing air exchange for the silica to effectively absorb moisture.

When a user needs to take a dose, the process begins with the removal of the upper cap 104. This action exposes the internal mechanism, which is the heart of the device's functionality. The upper cap 104, with its anti-leak design, plays a crucial role at this stage; when in place, it ensures the substance is securely contained, preventing any spillage or exposure to external elements. Once removed, it allows the user to access the substance delivery system. The next step involves the side dispenser knob 106, a key player in the dispensing process. As the user twists this knob 106, it dispenses the powdered substance.

It shall be appreciated by person skilled in the art that the term powdered substances include medications within its scope and the dispenser device disclosed herein can be efficiently used in form of a nasal inhaler device that administers powdered medication into nasal cavities. Furthermore, the scope of this disclosure includes other powdered substances such as salt, pepper and other powdered substances that are prone to damage due to moisture and the dispenser device claimed herein shall work equally for such powdered substances.

One or more components of the invention are described as unit for the understanding of the specification. Additional or less units can be included without deviating from the novel art of this disclosure. In addition, each unit can include any number and combination of sub-units, and systems, implemented with any combination of hardware and/or software units.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A dispenser device comprising:
a housing body;
an upper cap with an anti-leak feature that snugly fits onto the aluminium housing body, forming a seal to prevent the escape of powdered substance;
a side dispenser knob connected to an internal dispensing mechanism, the knob operable to release a predetermined amount of powdered substance from the dispenser device;
a bottom cap equipped with a compartment containing silica particles, the compartment being refillable and designed to absorb moisture within the dispenser device.

2. The dispenser device of claim 1, wherein the side-dispenser includes a sealing mechanism such as a gasket or an O-ring.

3. The dispenser device of claim 1, wherein the side dispenser knob, upon being twisted, releases the powdered substance.

4. The dispenser device of claim 1, wherein the compartment in the bottom cap is easily accessible for the replacement of silica particles and designed to minimize the ingress of moisture to the powdered substance.
